(19) United States
(12) Patent Application Publication
(10) Pub. No.: US 2005/0193448 A1
(43) Pub. Date: Sep. 1, 2005

(54) Methods for increasing one or more glucosinolates in a plant

(75) Inventors: Gary M. Gardner, Minneapolis, MN (US); Albert H. Markhart III, White Bear Lake, MN (US); Gerard Engelen-Eigles, St. Paul, MN (US); Lynette Y. Wong, St. Paul, MN (US); Carl J. Rosen, West St. Paul, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN (US)

(21) Appl. No.: 11/054,528

(22) Filed: Feb. 9, 2005

Related U.S. Application Data
(60) Provisional application No. 60/542,917, filed on Feb. 9, 2004.

(51) Int. Cl. A01H 5/00; A01H 3/00

(52) U.S. Cl. 800/306; 47/58.1 R

(57) ABSTRACT

The present invention provides methods for growing plants to result in increased concentrations of one or more glucosinolates. The plant may be a member of the order Capparales, such as a Brassicaceae, a Capparaceae, or a cultivar thereof. The methods include exposing a plant to an altered radiation condition, a water deficit condition, an altered growth temperature, an altered level of sulfur and/or nitrogen, or a combination thereof.

### CONTINUING APPLICATION DATA

This application claims the benefit of U.S. Provisional Application Ser. No. 60/542,917, filed Feb. 9, 2004, which is incorporated by reference herein.

### BACKGROUND

Phenethyl isothiocyanate occurs as its glucosinolate conjugate, gluconasturtiin, in a variety of cruciferous vegetables. Typical levels of gluconasturtiin are, in milligram (mg) per 100 gram (gm) fresh weight, approximately 8.5 in turnip root, 0.2-1.7 in cabbage, 2-26 in Chinese cabbage, and approximately 72 in watercress (Tookey et al., 1980, Glucosinolates, In: Toxic Constituents of Plant Stuffs. Second ed. (Ed: I. E. Liener,) Academic Press, New York, 103-142; Carlson et al., 1981, J. Agric. Food Chem. 29: 1235-1239; Chung et al., 1992, Cancer Epidemiol., Biomarkers & Prev. 1: 383-388). When these vegetables are chewed or otherwise macerated, the enzyme myrosinase catalyzes the hydrolysis of gluconasturtiin, releasing PEITC (Tookey et al., 1980, Glucosinolates, In: Toxic Constituents of Plant Stuffs. Second ed. (Ed: I. E. Liener,) Academic Press, New York, 103-142). The cancer chemopreventive properties of PEITC have been well documented in laboratory animals, and include, for instance, rat mammary tumorigenesis, mouse forestomach tumorigenesis, and mouse lung tumorigenesis induced by various agents.

Effects of light and temperature on PEITC content in crucifers have not been studied extensively and are unclear. Palaniswamy et al. (Palaniswamy et al., 1996, Hortscience, 34:578) demonstrated that irradiance and photoperiod interact to affect Nasturtium officinale R. Br. PEITC concentration. Specifically, PEITC content increased 60% when plants were exposed to a one week increase in irradiance from 265 to 434 μmol m⁻²s⁻¹ when grown under short-day but not long-day conditions. Increase in air temperature from 15° C. to 25° C. did not affect PEITC content in N. officinale (Palaniswamy et al., 1996, Hortscience, 34:578). In contrast, Raphanus salivurn L. ‘Burpee White’ isocyanate content decreased as mean daily air temperature increased from 14° C. to 22° C. (Bible and Chong, 1975, HortScience 10:484-485).

Several reports identify seasonal variation in glucosinolate concentration in crucifers (MacLeod and Nussbaum, 1977, Phytochemistry 16:861-865; Bible and Chong, 1975, HortScience 10:484-485; Bible et al., 1980, J. Amer. Soc. Hort Sci. 105:88-91; Rosa et al., 1996, J. Sci. Food Agric. 71:237-244). However, whether seasonal effects on glucosinolate concentration are due to differences in temperature, light, other environmental factors, or factors associated with plant maturity, is unclear.

Rosa et al. (1994, J. Sci. Food Agric., 66:457-463) demonstrated that glucosinolate concentration in Brassica oleracea var acephala ‘Galega’ and var. capitala ‘Predena’ varied during a 24 hour period. Glucosinolate concentration decreased between 1000 and 1400 hours. Highest glucosinolate concentrations were apparent at 1800 hours. A temperature interaction with circadian variation in glucosinolate content was obvious as a peak in glucosinolate concentration did not occur until 0600-1000 hours when plants were grown at reduced temperatures (specific temperatures not identified).

The importance of glucosinolates as flavor determinants (Fenwick et al., 1983, Critical Reviews in Food Science and Nutrition. CRC Press. Vol. 18(2), pp. 123-201), predator repellents or attractants (Harbome, 1993, Introduction to Ecological Biochemistry. Academic Press, New York, N.Y., pp 128-158), and as the precursor of anticarcinogenic compounds such as PEITC ( Fenwick et al., 1983, Critical Reviews in Food Science and Nutrition. CRC Press. Vol. 18(2), pp. 123-201) has prompted research on genotypic, diurnal, and environmental influences on content and composition of these important compounds. Several reports on the effect of planting date, growing season, and irrigation treatment suggest that plant water status is one determinant of the glucosinolate concentration in the plant tissue (MacLeod and Nussbaum, 1977, Phytochemistry 16: 861-865; Rosa et al., 1996, J. Sci. Food Agric. 71:237-244). These studies, however, concentrated on climatic conditions and only indirectly addressed the importance of plant water status. For example, in a study of 14 cabbage cultivars planted at several dates, Bible et al. (1980, J. Amer. Soc. Hort Sci. 105:88-91) found that late maturing cultivars or plants planted later in the season had higher glucosinolate levels than earlier maturing cultivars or plants planted earlier in the season. The authors suggested that this may be due to increased heat and water stress later in the summer. Plants grown with regular irrigation had lower glucosinolate levels than nonirrigated plants (Bible et al., 1980, J. Amer. Soc. Hort Sci. 105:88-91).

Adjustments in primary and secondary metabolism have been studied in a number of genera as mechanisms of acclimation to drought stress. Polyamine and polyhydroxylated sugar alcohol production increase in response to decreased water status in most plants (Rajam, 1997, Polyamines, In: Plant Ecophysiology ed. M.N.V. Prasad. John Wiley and Sons, N.Y. pp 343-374). Alterations in sulfur containing compounds are also likely, but much less studied.

In greenhouse studies withholding water from pot-grown watercress resulted in a 6.4 fold increase in phenethyl isothiocyanate (Freeman and Mossadeghi, 1973, J. Hort. Sci 48: 365-378). Cabbage plants grown in the field under rain out shelters also had substantial increases in flavor providing compounds when water stressed (Freeman and Mossadeghi, 1973). Unfortunately, the level of stress was not quantified, but was severe enough to reduce yield by almost 50%. Research that quantifies the effect of various conditions on PEITC concentrations and yield may provide information that will improve the pharmaceutical quality of these crops.

### SUMMARY OF THE INVENTION

The present invention is directed to methods for growing plants to result in increased concentrations of one or more glucosinolate such as, for instance, gluconasturtiin, glucobrassicin, glucotropaelin, or glucoraphanin. Typically, the plant is a member of the order Capparales, such as a Brassicaceae, a Capparaceae, or a cultivar thereof. Examples include, for instance, watercress, cabbage, Chinese cabbage, and turnip.

The present invention includes exposing the plant for at least about 3 days to a light photoperiod and a dark photoperiod and an altered radiation condition. The light photoperiod includes white light having photosynthetically active radiation from about 350 μmol/second/meter to about 500 μmol/second/meter, and the altered radiation condition includes extending the length of the light photoperiod, varying the spectral quality of the light, exposing the plant to radiation during the dark photoperiod, or a combination thereof. The plant exposed to the altered radiation condition has a concentration of a glucosinolate that is greater than a concentration of glucosinolate in a control plant that is exposed to white light for a light photoperiod of about 8 consecutive hours for each 24 hours. In one aspect, the altered radiation condition may include extending the length of the light photoperiod to between at least about 14 consecutive hours and about 18 consecutive hours for each 24 hour period. In another aspect, the altered radiation condition includes exposing the plant to red light, wherein the plant is exposed to the white light and the red light at the same time. In another aspect, the altered radiation condition includes extending the length of the light photoperiod to between at least about 14 consecutive hours and about 18 consecutive hours for each 24 hour period, and exposing the plant to red light, wherein the plant is exposed to the red light after exposure to the white light. In yet another aspect, the altered radiation condition includes exposing the plant during the light photoperiod to the white light for between at least about 6 consecutive hours and about 10 consecutive hours for each 24 hour period, and wherein the plant is exposed during the dark period to the white light for between at least about 5 minutes to about 15 minutes during each two hour interval of the dark period.

The present invention also includes exposing the plant to water deficit conditions until a leaf of the plant begins to wilt and then watering the plant, wherein the plant exposed to the water deficit conditions has a concentration of a glucosinolate that is greater than a concentration of the glucosinolate in a control plant not exposed to the water deficit conditions.

Another method of the present invention includes exposing the plant for about one week to a temperature of less than about 25° C., wherein the plant exposed to the temperature has a concentration of a glucosinolate that is greater than a concentration of the glucosinolate in a control plant exposed to a temperature of 25° C. or greater for about one week. In one aspect, the temperature of less than about 25° C. is substantially continuous during the week. In another aspect, the plant is exposed to a light period and a dark period, wherein the temperature during the light period is no greater than about 20° C. and the temperature during the dark period is no greater than about 15° C.

The present invention also includes exposing the plant to a composition having nitrogen and sulfur, wherein sulfur is applied at a rate that is about 100% greater than a recommended rate, wherein nitrogen is applied at a rate that is about 30% lower than a recommended rate. The plant exposed to such altered application rates has a concentration of a glucosinolate that is greater than a concentration of the glucosinolate in a control plant exposed to recommended rate or nitrogen and sulfur.

The terms “comprises” and variations thereof do not have a limiting meaning where these terms appear in the description and claims. Unless otherwise specified, “a,” “an,” “the,” and “at least one” are used interchangeably and mean one or more than one.

### BRIEF DESCRIPTION OF THE FIGURES

FIG. 1. Effect of long-day versus short-day light treatments on gluconasturtiin content. FW, fresh weight.

FIG. 2. Effect of enhancement with low doses of red light on gluconasturtiin content.

FIG. 3. Effect of end-of-day treatment with red light on gluconasturtiin content. EOD-R, end of day red light; EOD-FR, end of day far red; NAS, gluconasturtiin, FW, fresh weight.

FIG. 4. Effect of interrupting the night with brief periods of white light on gluconasturtiin content. NAS, gluconasturtiin, FW, fresh weight; SD&NB, short day and night bread treatment; SD&No NB, short day and night bread treatment.

FIG. 5. Effect of water deficit treatments on gluconasturtiin content. FW, fresh weight.

FIG. 6. Effect of growing plants at cooler temperatures on gluconasturtiin content. NAS, gluconasturtiin.

FIG. 7. Effect of growing plants at cooler night temperatures than day temperatures on gluconasturtiin content.

### DETAILED DESCRIPTION OF PREFERRED EMBODIMENTS OF THE INVENTION

The present invention is directed to methods for growing a plant to cause an increase in the concentration of one or more glucosinolates in the plant. The methods typically include providing a plant, and exposing the plant to a particular condition, where the plant exposed to the condition has a greater concentration of one or more glucosinolates when compared to the same type of plant that is not exposed to the same condition, i.e., a control plant. These methods typically do not cause an increase in biomass in the plant that is exposed to one or more of the conditions described herein, and often result in less biomass when compared to a plant that is not exposed to the same condition. Those skilled in the art do not typically grow plants under conditions that result in lower biomass production.

The plant is exposed to a 24-hour photoperiod that includes a light period and a dark period, and unless noted otherwise, the exposure to the 24-hour photoperiod is typically repeated for at least about 7 consecutive days. In general, the plant is harvested at the end of a repeated period of exposure. The amount of light used for biomass production that the plant is exposed to is referred to as photosynthetically active radiation (PAR). This term, also referred to in the art as photosynthetic photon flux density, is the total irradiance between 400 nanometers (nm) and 700 nm, and is expressed in the units “μmol s⁻¹m⁻².” This term is known in the art and is routinely used to describe the amount of radiation for photosynthesis to which a plant is exposed. PAR is typically measured by using a sensor at the top of the plant canopy in the center of the growing area.

In some aspects, the light to which a plant is exposed may include a spectral photon distribution that is similar to sunlight in that it includes wavelengths between about 360 nm and about 800 nm and is relatively uniform in energy throughout that range. This type of light is referred to herein as white light. White light may also be provided from a metal halide lamp, or a lamp having a spectrum similar to a metal halide lamp (see Gardner and Graceffo, 1982, Photochem. Photobiol., 36:349-354 for an example of the spectral photon distribution typically produced by a metal halide lamp).

In other aspects, the light to which a plant is exposed may include a spectral photon distribution that ranges from about 600 nm to about 700 nm, and optionally has a peak wavelength at about 650 nm to about 670 nm. This type of light is referred to herein as red (R) light. Red light may be provided by lamps having a spectra similar to that of Sylvania fluorescent lamps (F48T12/2364/HO) filtered through an Encapsulite red tube guard (Lighting Plastics of Minnesota, St. Louis Park, Minn., USA) (see Howe, et al., 1996, Physiologia Plantarum, 97:95-103 for spectra of these lamps with similar filters).

It is expected that the plant provided in the method can be at a developmental stage as young as the first mature leaf stage, and that there is no upper limit on the age of the plant. Typically, the plant will be at about the 4th or 5th mature leaf stage. The plant can be a member of the order Capparales, such as the Brassicaceae family (also referred to as the Cruciferae family) and the Capparaceae family (also referred to as the Caper family). Specific examples include watercress (Nasturtium officinale), cabbage, Chinese cabbage, and turnip, and the cultivars of these plants.

Glucosinolates that can be increased in the plants include, for instance, gluconasturtiin (2-phenylethylglucosinloate), glucobrassicin (3-indolylmethylglucosinolate), glucotropaelin (benzylglucosinolate), and glucoraphanin (4-(methylsulfinyl)butylglucosinolate). One or more of these may be increased. The concentration of one or more glucosinolates in a plant can be increased, typically by about 5%, preferably by about 10%, more preferably by about 15% or greater compared to the same type of plant that is not grown as described in the methods of the present invention. Methods for measuring the concentration of glucosinolates in plants are routine and known in the art (see, for instance, Lewke et al., 1996, Gartenbauwissenschaft. 61:179-183, and the Examples herein).

The condition to which a plant is exposed in the methods of the present invention typically includes exposing the plant to an altered radiation condition, altering the growth temperature, altering the water status of the plants, altering the levels of sulfur and/or nitrogen in the surrounding growth medium (for instance, soil), or a combination of any two or more of these conditions. Altered radiation conditions include, for instance, extending the length of the light photoperiod, varying the spectral quality of the light, exposing the plant to radiation during the dark photoperiod, or a combination thereof. In one aspect, plants are exposed to a 24-hour photoperiod including from at least about 14 to about 18 hours of white light, preferably about 16 hours white light. The remaining portion of the 24-hour photoperiod is dark. In this aspect of the invention, the plant is exposed to PAR between at least about 350 μmol s⁻¹m⁻² to about 50 μmol s⁻¹m⁻², preferably about 400 μmol s⁻¹m⁻².

In another aspect of the invention, plants are exposed to a 24-hour photoperiod including from at least about 14 to about 18 hours of white light, preferably about 16 hours of white light. The remaining portion of the 24-hour photoperiod is dark. In this aspect of the invention the spectral quality of the light is enriched with red light The PAR is between at least about 350 μmol s⁻¹m⁻² to about 450 μmol s⁻¹m⁻², preferably about 400 μmol s⁻¹m⁻², where the red light lamps provide about 2-8 % of the total PAR.

In yet another aspect of the present invention, a plant is exposed to a 24-hour photoperiod including from about 14 to about 18 hours of white light, preferably about 16 hours of white light. The PAR is between at least about 350 μmol s⁻¹m⁻² to about 450 μmol s⁻¹m⁻², preferably about 400 μmol s⁻¹m⁻². At the end of each light period, the plant is further exposed to red light, preferably for between at least about 2 minutes to about 10 minutes, more preferably, about 5 minutes.

The methods of the present invention also include exposing a plant to a 24 hour photoperiod including from at least about 6 to about 10 hours of white light, preferably about 8 hours of white light. The PAR may be between at least about 400 μmol s⁻¹m⁻² to about 500 μmol s⁻¹m⁻², preferably about 450 μmol s⁻¹m⁻². During the dark period of the 24 hour photoperiod, the plant is exposed to a brief pulse of white light. Preferably, the pulse may last from about 5 minutes to about 15 minutes, more preferably, about 10 minutes, and the pulse may occur multiple times during the dark period, typically once every about 2 hours. The exposure to the 24 photoperiod is typically repeated over consecutive days, preferably for at least about 14 consecutive days.

In another aspect, the plant is exposed to water deficit conditions to increase the level of one or more glucosinolates. Water is reduced or withheld from the plant until visible signs of wilt are present in the youngest leaves of the plant. The plant may then be harvested while it is displaying wilting, or it may be watered until it is fully turgid and then harvested. Optionally, the plant may be watered daily for consecutive days, for instance, a total of about 3 days, and not watered again until visible signs of wilt are present in the youngest leaves of the plant. The plant may be watered again, and it may be harvested at least about 24 hours after watering. The process of reducing or not watering until wilt followed by watering may be repeated 2 or more times.

The level of one or more glucosinolates may be increased in a plant by exposure to cooler temperatures. Plants are typically grown at higher temperatures to maximize the biomass production by the plant. The plant is grown at a temperature that is less than about 25° C., preferably, less than about 20° C., more preferably, less than about 15° C. Alternatively, the plant may be exposed to a cooler temperature at night. In this aspect, the plant may be exposed to a temperature at night that is lower than the daytime temperature, preferably, about 5° C. to about 10° C. lower. For instance, a plant can be exposed to about 20° C. during the day and about 15° C. to about 10° C. during the night. In these aspects of the invention, the 24-hour photoperiod may have a light period of between about 8 hours to about 18 hours of white light, preferably about 16 hours of white light. The PAR may be between at least about 350 μmol s⁻¹m⁻² to about 500 μmol s⁻¹m⁻², preferably about 450 μmol s⁻¹m⁻².

Another aspect of the invention includes increasing the level of one or more glucosinolates in a plant by adjusting the application rates of nitrogen and sulfur. In this method, fertilizer is applied to the plants at a rate below the recommended nitrogen application rate and above the recommended sulfur application rate. The fertilizer application rates vary depending upon the type of plant and the type of soil in which the plant is grown; however, these values are known in the art and readily available (see, for instance, Rosen and Eliason, 1996, Univ. Minn. Ext. Serv. BU-5886-E). For instance, when the plant is cabbage and it is present in a soil where the recommended nitrogen and sulfur application are about 200 kilogram per hectare (kg/ha) nitrogen and about 35 kg/ha sulfur, decreasing nitrogen to about 110 kg/ha and increasing sulfur to about 50 kg/ha or about 100 kg/ha increased the levels of one or more glucosinolates in the cabbage. In general, the application rate of nitrogen is decreased by about 30% of the recommended nitrogen rate, and the application rate of sulfur is increased by about 100% of the recommended sulfur rate. In general, the skilled person can determine the recommended application rate of nitrogen and sulfur by evaluating the properties of the soil in which the plants are to be grown, and then determining the recommended application rate of nitrogen and sulfur by reference to known sources of such information (Rosen and Eliason, 1996, Univ. Minn. Ext. Serv. BU-5886-E).

A plant may be grown using one of the methods of the present invention, or grown using a combination of two or more of the methods. It is expected that combining two or more of the present methods will result in an increase in one or more glucosinolates that is at least additive. For instance, exposing a plant to a 24 hour photoperiod of about 16 hours of light and water deficit conditions resulted in an increase in gluconasturtiin that was additive. The present invention also includes methods for reducing the risk of cancer in an individual, for instance, a human, by feeding to the individual a plant grown using one or more of the methods described herein. Various animal models are available for determining whether feeding to an individual a plant grown using one or more of the methods described herein will reduce the risk of cancer. The study of chemopreventive properties of plants in animals (for instance, mice and rats) is a commonly accepted model for human disease. Also included in the present invention is a method of preparing a food using a plant grown using one of the methods described herein.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Comparison of Long-Day and Short-Day Treatments

Watercress (Johnny's Selected Seeds, Albion, Me.) was seeded in 12.5 cm square pots in moist soil-less media (SunGro Horticulture, SunShine SB-300 Universal, Bellevue, Wash.) containing sphagnum peat, bark, perlite, and vermiculite and grown in two EGC growth chambers (Model GCW-15, Environmental Growth Chambers, Chagrin Falls, Ohio). Emerging seedlings were grown at constant 20° C. and under long days (LD, 16 hours light, 8 hours dark). During the light period, the photosynthetically active radiation (PAR) was 450 μmol s⁻¹m⁻². PAR was measured with an Apogee Quantum Meter, Model QMSW-SS (Apogee Instruments, Inc., Logan, Utah). Plantlets were thinned to one plant per pot one week after seedling emergence. When plantlets reached the 5^{th} mature leaf stage (approximately 14 day from seeding), 30 plants were exposed to long days (LD) of 16 hours light and 8 hours dark, and 30 plants, in a separate chamber, were exposed to short days (SD, 8 hours light, 16 hours dark.). The light source consisted of 6 Sylvania 400 W metal halide (MH) lamps (Osram Sylvania Products, Inc., Manchester, N.H.), delivering PAR=400 μmol s⁻¹m⁻². Temperature was constant at 20° C. day and night. Plants were watered daily and fertilized every two days with 100 parts per million (ppm) nitrogen (N) by 15-5-1 5-Ca&Mg Peters Exel Water Soluble Fertilizer (Scotts-Sierra Horticultural Products Company, Marysville, Ohio). All above ground tissue of sample plants was frozen in liquid nitrogen and stored at −80° C. at the start of the photoperiod experiment and after 1 and 2 weeks of exposure to LD or SD.

Glucosinolate extraction. Frozen watercress samples were ground using a mortar and pestle that had been pre-chilled using liquid nitrogen. The ground plant material was transferred into rapidly boiling HPLC grade water and boiled for 10 minutes. The plant extract was filtered with a Whatman #1 (9 cm) filter paper, and the filtered extract was brought up to a final volume of 20 ml g⁻¹ fresh weight of tissue. The aqueous extract was mixed thoroughly, and aliquots of 15 ml were taken in small glass vials for ammonium sulfate precipitation. Extracts with ammonium sulfate were left overnight at 4° C. and then centrifuged at 10,000 g for 30 minutes. The aqueous extract was filtered using a 0.2 μm syringe fitted filter unit (Nalge Company, Rochester, N.Y.). The supernatant was stored at −20° C. prior to analysis by high-pressure liquid chromatography (HPLC).

HPLC. Glucosinolates were separated and quantified by reverse phase HPLC on a Waters system (Model 6000A, Waters Associates, Milford, Mass.) equipped with a Model 712 WISP autosampler and a UV detector (Model 783A, Applied Biosystems, Foster City, Calif.) set at 235 nm. Separation was accomplished using an HPLC protocol (modified from Lewke et al., 1996, Gartenbauwissenschaft. 61:179-183) using a Restek Ultra Aqueous C₁₈ analytical column (4.6 mm×15 cm, 5 μm) (Restek USA, Bellefonte, Pa.) with a flow rate of 1.5 mL/minute. Two different mobile phases were chosen; A: 0.1 M ammonium acetate and B: 30% methanol in 0.1M ammonium acetate. The elution protocol was: 0 to 4 min., 100% A; 4 to 14 min., linear gradient to 70% B; 14 to 17 min., linear gradient to 100% B for 5 min., 22 to 23.5 min., linear gradient to 100% A for 15.5 minutes post-run. Gluconasturtiin was identified by coelution with a known sample of phenethyl glucosinolate potassium salt (catalog number P2502, LKT Laboratories, St. Paul, Minn.).

In all experiments data were taken on fresh weight (FW) and gluconasturtiin (NAS) content. Data were analyzed by analysis of variance using the Statistix (St. Paul, Minn.) program.

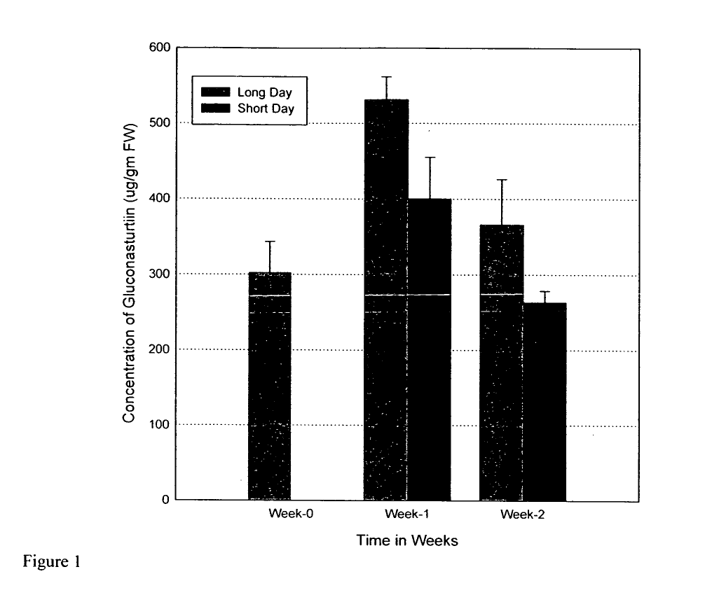

Results. Plants grown under long days had significantly greater gluconasturtiin content, on a fresh weight basis, than plants grown under short days (FIG. 1).

### Example 2

### Increase in Gluconasturtuin Content by Enhancenient with Low Doses of Red Light

Watercress plants were grown at 20° C. under LD (16 hours) to the 5^{th} mature leaf stage as described for Example 1. When this stage was reached, the plantlets were moved to different EGC growth chambers: one with MH lamps enriched with far-red (FR) light, and one with MH lamps enriched with red (R) light; total PAR=400 μmol s⁻¹m⁻². The R light was provided by three R fluorescent lamps (Sylvania F48T12/2364/HO) filtered through an Encapsulite red tube guard (Lighting Plastics of Minnesota, St. Louis Park, Minn., USA). The FR light was provided by six FR fluorescent lamps (Sylvania F48T12/232/HO) filtered through an Encapsulite FR tube guard (Lighting Plastics of Minnesota, St. Louis Park, Minn., USA). All chambers were adjusted to equal PAR at the level of the plants by altering the distance between the lights and the plants. In the R chamber, the R lamps provided about 2% of the total PAR, and the contribution of the FR lamps to PAR was, as expected, negligible (about 0.1%). The total irradiance of the R lamps was approximately equal to the total irradiance of the FR lamps. The irradiance and spectral quality of the R and FR lamps were measured with an Apogee Model SPEC-UV/PAR Spectroradiometer (Apogee Instruments, Inc., Logan, Utah).

The R and FR lamps were switched on at the same time as the ME lamps. The R and FR lamps were switched off I minute after the MH lamps because the MH lamps emitted a glimmer of light for about 30 s after being switched off. Plants were watered and fertilized as described for Example 1 and grown under a LD photoperiod (16 hours light; 8 hours dark). Individual watercress plants were harvested after 1 and 2 weeks of variation in light quality.

Glucosinolate extraction. Glucosinolate was extracted and stored as described in Example 1.

HPLC. Glucosinolates were separated and quantified by reverse phase HPLC as described in Example 1.

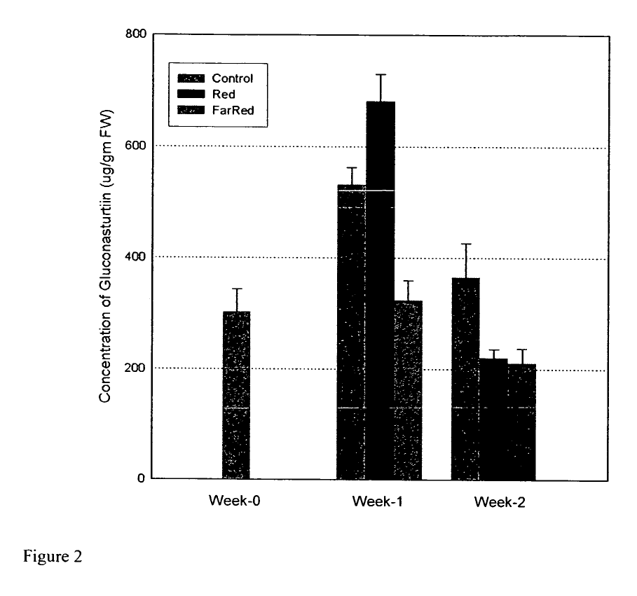

Results. Plants grown under metal halide lamps (white light) showed an increase in gluconasturtiin content with red-light supplementation from fluorescent lamps after one week (FIG. 2). This increase did not occur if the metal halide light was enriched with far-red light. Since the light in both chambers was normalized to the same PAR, this effect cannot be attributed to increased photosynthesis under the supplemental red-light treatment.

### Example 3

### Increase in Gluconasturtiin Content by an End-of-Day Treatment with Red Light

Watercress plants were grown at 20° C. under LD (16 hours) to the 5^{th} mature leaf stage as described for Example 1. When this stage was reached, the plantlets were moved to different EGC growth chambers as described for Example 2: one with MH lamps enriched with far-red (FR) light, and one chamber with MH lamps enriched with red (R) light; total PAR=300 μmol s⁻¹m⁻². The R light was provided by three R fluorescent lamps (Sylvania F48T1 2/2364/HO) filtered through an Encapsulite red tube guard (Lighting Plastics of Minnesota, St. Louis Park, Minn., USA). The FR light (radiation predominantly in the range from 700-800 nm) was provided by six FR fluorescent lamps (Sylvania F48T12/232/HO) filtered through an Encapsulite FR tube guard (Lighting Plastics of Minnesota, St. Louis Park, Minn., USA). Since FR light is not measured by the quantum sensor used to measure PAR, the FR was measured with a spectroradiometer able to measure the FR wavelengths. The FR was adjusted to be about the same irradiance as the R, about 8-10 μmol s⁻¹m⁻².

Plants were watered and fertilized as described for Example 1 and grown under a LD photoperiod (16 hours light; 8 hours dark) under metal halide lamps (only). At the end of the 16 hours white light day, plants were given either five minutes of red light or ten minutes of far-red light. Individual watercress plants were harvested after 1 and 2 weeks of different end-of-day treatments.

Glucosinolate extraction and analysis were carried out as for Examples 1 and 2.

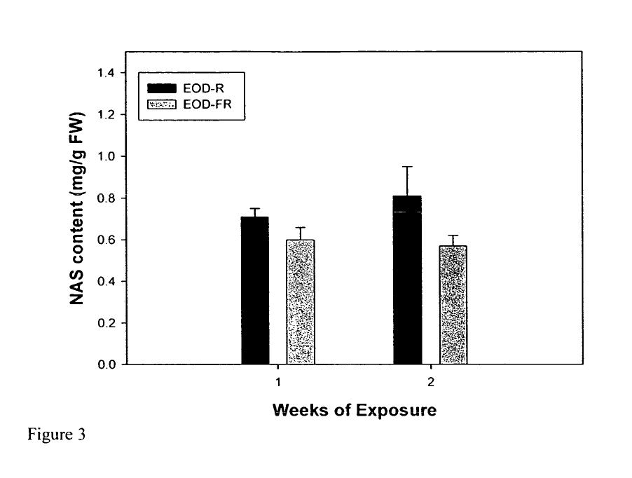

Results. An end-of-day treatment with red light resulted in an increase in gluconasturtiin concentration (FIG. 3). This increase persisted throughout the two weeks of the experiment. It did not occur if far-red light was used for the end-of-day treatment.

### Example 4

### Increase in Gluconasturtiin Content by Interrupting the Night with Brief Periods of White Light

Watercress plants were grown at 20° C. under LD (16 hours) to the 5^{th} mature leaf stage as described for Example 1. When this stage was reached, the plantlets were moved to different EGC growth chambers as described for Example 1, with MH lamps, total PAR=450 μmol s⁻¹m⁻². Plants were watered and fertilized as described for Example 1 and grown under a SD photoperiod (8 hours light; 16 hours dark) under metal halide lamps (only). Plants in one chamber were not given additional light. Plants in a second chamber were given ten minutes of MH light every two hours during the 16-hour night. Individual watercress plants were harvested after 1 and 2 weeks with or without night break treatments.

Glucosinolate extraction and analysis were carried out as for Examples 1 and 2.

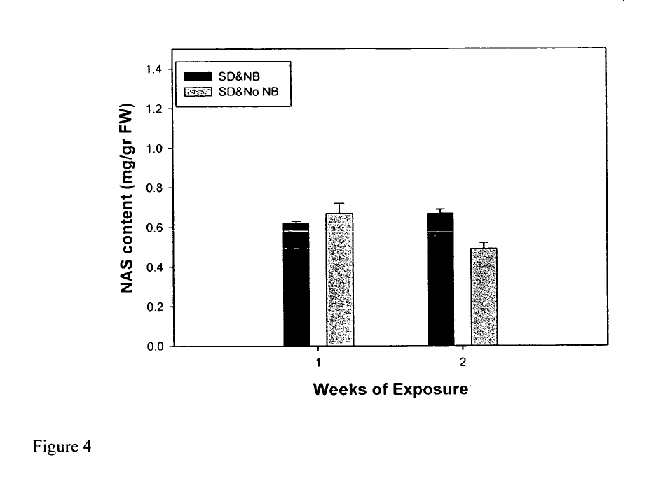

Results. Interruption of the dark night every two hours with white light causes an increase in gluconasturtiin concentration after two weeks of treatment, relative to a short-day control (FIG. 4).

### Example 5

### Water Deficit Treatments

Watercress (Johnny's Selected Seeds, Albion, Me.) was seeded in 12.5 cm square pots filled with a 1:1 ratio of sand and Turface MVP media (Profile Products LLC, Buffalo Grove, Ill.) in a growth chamber (EGC Model GC-36) maintaining a 16-hour photoperiod using Sylvania 400 W metal halide lamps (Osram Sylvania Products, Manchester, N.H.; PAR=450 μmol m⁻²s⁻¹) at a growth temperature of 20° C. At the 5^{th} mature leaf stage, treatments were randomly assigned to plants. All plants assigned to the water stress treatment were not watered until visible signs of wilt in the youngest leaves were present and then all plants were watered daily for three days. All above ground tissue was harvested at the second cycle of stress (i.e., after the leaves were allowed to wilt, rehydrated daily for 3 days, allowed to wilt, and then rehydrated daily for 3 days again) and at 72 hrs post rehydration. Tissue was immediately frozen in liquid nitrogen and stored at −80° C.

Glucosinolate extraction. Frozen plant material was extracted by boiling in HPLC grade water for 10 minutes. After passing the plant extract through a Whatman #1 paper filter, the extract was brought up to a final volume of 20 mL/g of fresh weight tissue. Aliquots of 15 mL were removed and protein was precipitated by adding 2.8 g solid ammonium sulfate/5 ml of extract. Vials were incubated overnight at 4° C. Samples were filtered using a 0.2 μm syringe driven filter unit (Nalge Company, Rochester, N.Y.). The supernatant was stored at −20° C.

HPLC analysis Glucosinolates were separated and quantified by reverse phase HPLC on a Waters system (Model 6000A, Waters Associates, Milford, Mass.) equipped with a Model 712 WISP autosampler and a UV detector (Model 783A, Applied Biosystems, Foster City, Calif.) set at 235 nm. Separation was accomplished using an HPLC protocol (modified from Lewke et al., 1996, Gartenbauwissenschalt. 61:179-183) using a Restek Ultra Aqueous C₁₈ analytical column (4.6 mm×15 cm, 5 μm) (Restek USA, Bellefonte, Pa.) with a flow rate of 1.5 mL/minute. Two different mobile phases were chosen; A: 0.1 M ammonium acetate and B: 30% methanol in 0.1M ammonium acetate. The elution protocol was: 0 to 4 min., 100% A; 4 to 14 min., linear gradient to 70% B; 14 to 17 min., linear gradient to 100% B for 5 min., 22 to 23.5 min., linear gradient to 100% A for 15.5 minutes post-run. Gluconasturtiin was identified by coelution with a known standard (LKT Laboratories, St. Paul, Minn.).

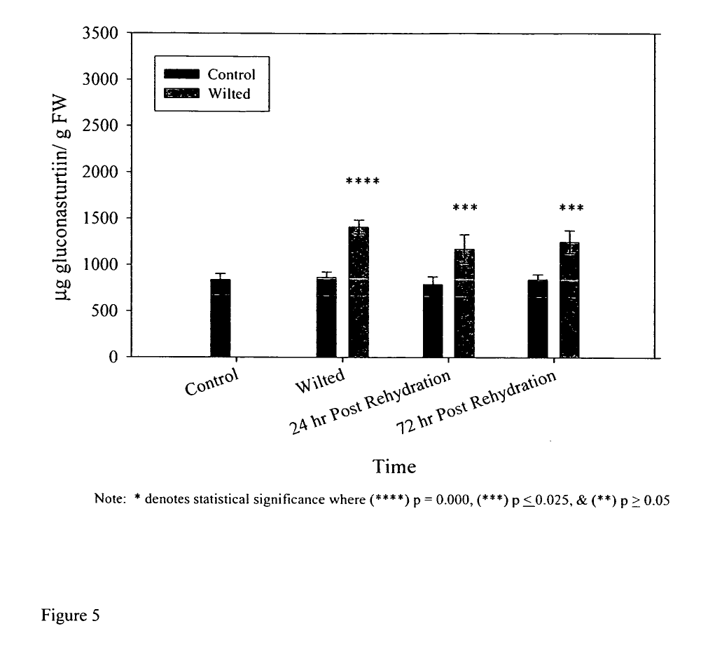

Results. Water deficit can increase the level of gluconasturtiin in watercress, and increasing the number of cycles of water deficit further increased the levels of gluconasturtiin (FIG. 5). Furthermore, this increase in gluconasturtiin is maintained if the stressed plants are re-watered until turgid.

### Example 6

### Increase in Gluconasturliin Content by Growing Plants at Cooler Temperatures

Watercress plants were grown at 20° C. under LD (16 hours) to the 5^{th} mature leaf stage as described for Example 1. When this stage was reached, the plantlets were moved to different growth chambers: one at continuous 15° C., one at continuous 20° C., and one at continuous 25° C. The photoperiod was 16 h using metal halide lamps; PAR=450 μmol s⁻¹s⁻¹m⁻². Watercress plants were watered and fertilized as described for Example 1. Individual plants were weighed (fresh weight) and frozen after 1 or 2 weeks of growth at the indicated temperature.

Glucosinolate extraction and analysis were carried out as for Examples 1 and 2.

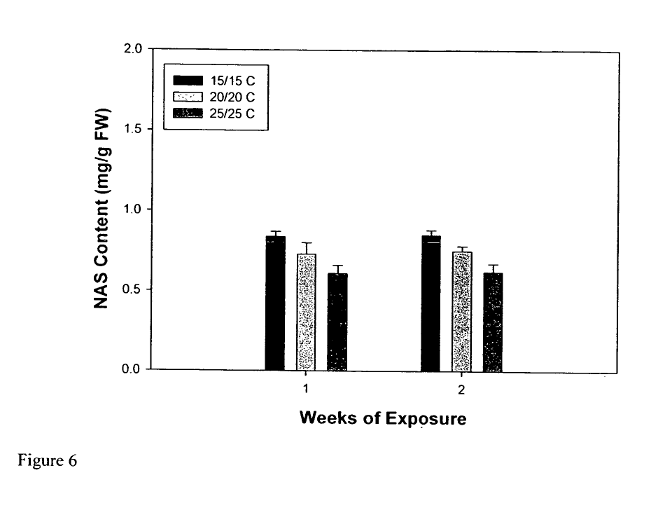

Results. Gluconasturtiin concentration was increased by growing plants at cooler temperature (FIG. 6).

### Example 7

### Increase in Gluconasturtiin Content by Growing Plants at Cooler Night Temperatures than Day Temperatures

Watercress plants were grown at 20° C. under LD (16 hours) to the 5^{th} mature leaf stage as described for Example 1. When this stage was reached, the plantlets were moved to different growth chambers: one at continuous 20° C. and one at a day temperature of 20° C. and a night temperature of 15° C. The photoperiod was 16 h light/8 h dark using metal halide lamps; PAR=450 μmol s⁻¹m⁻². Watercress plants were watered and fertilized as described for Example 1. Individual plants were weighed (fresh weight) and frozen after 1 or 2 weeks of growth at the indicated temperature.

Glucosinolate extraction and analysis were carried out as for Examples 1 and 2.

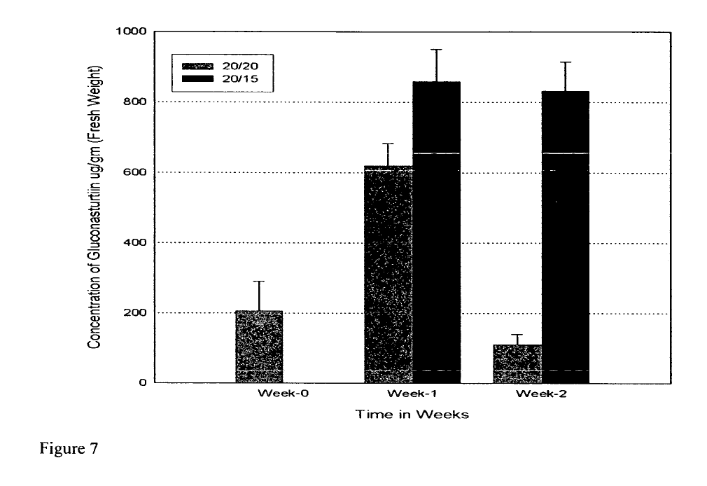

Results. Growing plants under cooler night temperatures resulted in higher gluconasturtiin concentrations (FIG. 7). In other experiments, a night temperature of 10° C. (with a day temperature of 20° C.) also resulted in higher gluconasturtiin levels than a continuous growth temperature of 20° C.

### Example 8

### Increasing Glucobrassicin Content in Cabbage by Adjusting Sulfur and Nitrogen Fertilizer Application Rates

A field study to determine the effect of nitrogen and sulfur fertilizer on glucobrassicin content of cabbage was conducted at the Sand Plain Research Farm in Becker, Minn., on a Hubbard loamy sand soil. The previous crop grown was rye, and selected soil properties before planting were as follows (0-15 cm): organic matter, 2.1%; pH (1 soil:1 water) 5.9; Bray P, 24 ppm; ammonium acetate extractable K, Ca, Mg, 103, 809, 151 ppm respectively; sulfate-S, 6 ppm; DTPA Zn, Fe, Cu, Mn, 0.5, 27, 0.5, 8 ppm respectively; hot water extractable boron, 0.3 ppm. Nitrate-N in the top 60 cm of soil was 13 lb/A. Prior to planting, 20 kg/ha P, 160 kg/ha K, and 3 kg/ha B were applied and then incorporated to a depth of 15 cm with a field cultivator. ‘Grand Slam’ a green cabbage and ‘Vorox’ a red cabbage (Jordan Seed, Woodbury, Minn.) were seeded in 50 count transplant flats containing moist soiless seeding media (SunGro Horticulture, SunShine SB-300 Universal, Bellevue, Wash.) and grown in the greenhouse on the St. Paul campus at the University of Minnesota for five weeks. Transplants were planted at Becker the first week of July. A transplant solution was prepared with Diazinon at 31 mL per 200 L to control root maggots and 11 -48-8 at 1.4 kg per 200 L as a starter fertilizer. Approximately 250 mL of the solution was applied to the base of the plants after setting in the field. All planting operations were done by hand.

Six fertilizer treatments tested were: 1) 110 kg/ha N and 0 kg/ha S; 2) 110 kg/ha N and 55 kg/ha S; 3) 110 kg/ha N, 110 kg/ha S; 4) 220 kg/ha N and 0 kg/ha S; 5) 220 kg/ha N and 55 kg/ha S; 6) 220 kg/ha N, 110 kg/ha S. The recommended nitrogen and sulfur fertilizer application rates for cabbage on this soil are 200 kg/ha N and 35 kg/ha S. The treatments were set out as a split plot design with four replications. Fertilizer was the main plot and cabbage cultivar was the subplot. Each main plot consisted of four rows 6 meters (m) in length with the two varieties randomized in the middle of the four rows. The first and fourth rows were border rows. Spacing within rows was 0.5 m and between rows was 1 m. Urea was used as the N source for the zero sulfur treatments and ammonium sulfate was used as the N and S source for the N and S treatments. Urea was used to equalize N application as needed in the N and S treatments. Nitrogen and sulfur fertilizers were applied in three equal applications at one, three, and five weeks after planting. Irrigation was applied after each fertilizer application with a solid set sprinkler system. Irrigation was applied as needed to prevent water stress. Grand Slam was harvested the second week in September, and Vorox was harvested the first week in October. At harvest, the middle 8 heads from each harvest row were cut, trimmed, and weighed. One head from each plot was stored at 2° C. for at most two weeks until glucosinolate extraction could be performed.

Glucosinolate extraction. Each head in cold storage was weighed. One quarter of the head was placed into three times the weight per volume of boiling water and boiled for 5 minutes. The hot cabbage was placed in a blender and ground for 2 minutes. One hundred milliliter (ml) aliquots were stored at −20° C. until analysis could be performed. Five ml of the cabbage blend was homogenized for 2 minutes using a Janke-Kundel Turrax T 25 homogenizer set at 12,000 rpm, at 4° C. The homogenate was then centrifuged at 4000 rpm, 4° C.

Strong Anion Exchange (SAX) solid phase extraction cartridges (500 mg, Supelco, Sigma Corp., St. Louis, Mo.) were used to bind the glucosinolates and desulphate them. First, the columns conditioned with 2 mL of 0.5 M sodium acetate, pH 4.6; followed by 2 mL of water. Next, 500 μL of cabbage supernatant was added to the SAX columns, followed by 1 mL of 0.02 M sodium acetate, pH 4.0. Next, 1 mL of sulfatase solution (0.2 mg/mL water or 3.1 units/mL water; Sigma S-9626; St. Louis, Mo.) was added and allowed to react overnight. The next day, 3 mL water was added and collected. The eluant volume was measured. This collection was either analyzed by HPLC or stored at −20° C. for later analyses. Further washing of the columns showed no desulphated glucosinolates. A sinigrin standard curve (Cat. # 85440, Sigma Corp., St. Louis, Mo.) was made up in water in separate tubes at concentrations from 0.6 to 12.2 μg/mL. A quarter of the sulfatase was added and it was allowed to react overnight. The next day, 100 μL of acetonitrile was added to stop the reaction and the solution was centrifuged in Amicon Filters (cat. # 4101, Millipore, Bedford, Mass.). The filtered solution was analyzed on HPLC.

HPLC Analyses. The analyses were done on a Waters 510 pump (Waters Corp.; Milford, Mass.), a Hewlett-Packard HP-1100 autosampler (Wilmington, Del.), and Shimadzu SPD-10A vp variable wavelength detector (Wilmington, Del.) set at λ=229 nm with a range 2.0. The column was a Phenomenex Luna C18, 5 μm spherical bead, 250×4.6 mm (Torrance, Calif.). The gradient conditions were as follows: solvent A; water and solvent B; acetonitrile; 0 to 2 min., 5% B to 15% B; 2 to 30 min., 15% B to 65% B; 30to 35 min., 65% B to 90% B; 35 to 37 min., 90% B to 5% B; and 37 to 60 min., 5% B. The flow rate was 1 mL/min. and 100 μL of each sample (the 3 mL SAX collection) was injected.

The data were collected and integrated on Peak Simple software (SRI Inc., Las Vegas, Nev.). The desulphated glucosinolates were identified by retention time, using standards kindly provided as a gift from Richard Mithen in Norwich, UK. The amounts of each desulphated glucosinolate was calculated based on a desulphated sinigrin standard curve and the published response factors relative to the desulphated sinigrin (Official Journal of the European Communities; L170, 03.07.27-34, 1990; Lewis, J., & Fenwick, G. R., Glucosinolate Content of Brassica Vegetables—Chinese Cabbages Pe-tsai (Brassica pekinensis ) and Pak-choi (Brassica chinensis), J. Sci. Food Agric., 45:379-386, 1988). The desulphated glucosinolate peaks were later confirmed by UV spectroscopy using a Waters Corp. 996 photodiode array detector.

All glucobrassicin data are expressed on fresh weight (FW) basis. Data were analyzed by analysis of variance using the SAS program (SAS/STAT user's guide, 4th edn. SAS Institute, Inc., N.C.).

Results. Plants grown with high sulfur and low nitrogen fertilizer maximized glucobrassicin content on a fresh weight basis in both cabbage cultivars (Table 1).

TABLE 1
Nitrogen, sulfur, and cultivar effects
on glucobrassicin content of cabbage.

| Treatments | | | |
|---|---|---|---|
| | N | S | Glucobrassicin |
| Variety | rate | rate | concentration (mg/kg FW) |
| Grand | 110 | 0 | 329 |
| Slam | | | |
| | 110 | 50 | 326 |
| | 110 | 100 | 414 |
| | 220 | 0 | 193 |
| | 220 | 50 | 296 |
| | 220 | 100 | 362 |
| Vorox | 110 | 0 | 1003 |
| | 110 | 50 | 1171 |
| | 110 | 100 | 1203 |
| | 220 | 0 | 707 |
| | 220 | 50 | 879 |
| | 220 | 100 | 907 |
| Statistics: Main effects | | | |
| Variety | Grand Slam | | 320 |
| | Vorox | | 978 |
| Significance | | | ** |
| N rate | 110 | | 741 |
| | 220 | | 557 |
| Significance | | | ++ |
| S rate | 0 | | 558 |
| | 50 | | 668 |
| | 100 | | 721 |

| | |
|---|---|
| Significance | |
| Linear | * |
| Quadratic | NS |
| Interactions | |
| Variety × N rate | ++ |
| Variety × S rate | NS |
| N rate × S rate | NS |
| N rate × linear S | NS |
| N rate × quadratic S | NS |
| Variety × N rate × S rate | NS |

NS = Non significant;
++, *, and ** refer to significant at 10%, 5% and 1% respectively.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. A method for growing a plant comprising:
providing a plant that is a member of the order Capparales;
exposing the plant for at least about 3 days to a light photoperiod and a dark photoperiod and an altered radiation condition, wherein the light photoperiod comprises white light comprising photosynthetically active radiation from about 350 μmol/second/meter to about 500 μmol/second/meter, wherein the altered radiation condition comprises extending the length of the light photoperiod, varying the spectral quality of the light, exposing the plant to radiation during the dark photoperiod, or a combination thereof, and wherein the plant exposed to the altered radiation condition has a concentration of a glucosinolate that is greater than a concentration of glucosinolate in a control plant that is exposed to white light for a light photoperiod of about 8 consecutive hours for each 24 hours.

2. The method of claim 1 wherein the altered radiation condition comprises extending the length of the light photoperiod to between at least about 14 consecutive hours and about 18 consecutive hours for each 24 hour period.

3. The method of claim 1 wherein the altered radiation condition comprises exposing the plant to red light, wherein the plant is exposed to the white light and the red light at the same time.

4. The method of claim 1 wherein the altered radiation condition comprises extending the length of the light photoperiod to between at least about 14 consecutive hours and about 18 consecutive hours for each 24 hour period, and exposing the plant to red light, wherein the plant is exposed to the red light after exposure to the white light.

5. The method of claim 1 wherein the altered radiation condition comprises exposing the plant during the light photoperiod to the white light for between at least about 6 consecutive hours and about 10 consecutive hours for each 24 hour period, and wherein the plant is exposed during the dark period to the white light for between at least about 5 minutes to about 15 minutes during each two hour interval of the dark period.

6. The method of claim 1 wherein the plant is a Brassicaceae, a Capparaceae, or a cultivar thereof.

7. The method of claim 6 wherein the plant is watercress, cabbage, Chinese cabbage, turnip, or a cultivar thereof.

8. The method of claim 1 wherein the glucosinolate is gluconasturtiin, glucobrassicin, glucotropaelin, glucoraphanin, or a combination thereof.

9. The method of claim 1 wherein the glucosinolate is increased by at least 5%.

10. A method for growing a plant comprising:
providing a plant that is a member of the order Capparales;
exposing the plant to water deficit conditions until a leaf of the plant begins to wilt;
watering the plant daily for three days, wherein the plant exposed to the water deficit conditions has a concentration of a glucosinolate that is greater than a concentration of the glucosinolate in a control plant not exposed to the water deficit conditions.

11. The method of claim 10 wherein the plant is a Brassicaceae, a Capparaceae, or a cultivar thereof.

12. The method of claim 11 wherein the plant is watercress, cabbage, Chinese cabbage, turnip, or a cultivar thereof.

13. The method of claim 10 wherein the glucosinolate is gluconasturtiin, glucobrassicin, glucotropaelin, glucoraphanin, or a combination thereof.

14. The method of claim 10 wherein the glucosinolate is increased by at least 5%.

15. A method for growing a plant comprising:
providing a plant that is a member of the order Capparales;
exposing the plant for about one week to a temperature of less than about 25° C., wherein the plant exposed to the temperature has a concentration of a glucosinolate that is greater than a concentration of the glucosinolate in a control plant exposed to a temperature of 25° C. or greater for about one week.

16. The method of claim 15 wherein the temperature of less than about 25° C. is substantially continuous during the week.

17. The method of claim 15 wherein the plant is exposed to a light period and a dark period, wherein the temperature during the light period is no greater than about 20° C. and the temperature during the dark period is no greater than about 15° C.

18. The method of claim 15 wherein the plant is a Brassicaceae, a Capparaceae, or a cultivar thereof.

19. The method of claim 18 wherein the plant is watercress, cabbage, Chinese cabbage, turnip, or a cultivar thereof.

20. The method of claim 15 wherein the glucosinolate is gluconasturtiin, glucobrassicin, glucotropaelin, glucoraphanin, or a combination thereof.

21. The method of claim 15 wherein the glucosinolate is increased by at least 5%.

22. A method for growing a plant comprising:
exposing a plant that is a member of the order Capparales to a composition comprising nitrogen and sulfur, wherein sulfur is applied at a rate that is about 100% greater than a recommended rate, wherein nitrogen is applied at a rate that is about 30% lower than a recommended rate, and wherein the plant exposed to such altered application rates has a concentration of a glucosinolate that is greater than a concentration of the glucosinolate in a control plant exposed to recommended rate or nitrogen and sulfur.

23. The method of claim 22 wherein the plant is a Brassicaceae, a Capparaceae, or a cultivar thereof.

24. The method of claim 23 wherein the plant is watercress, cabbage, Chinese cabbage, turnip, or a cultivar thereof.

25. The method of claim 22 wherein the glucosinolate is gluconasturtiin, glucobrassicin, glucotropaelin, glucoraphanin, or a combination thereof.

26. The method of claim 22 wherein the glucosinolate is increased by at least 5%.
